(19) United States
(12) Patent Application Publication
(10) Pub. No.: US 2018/0348053 A1
(43) Pub. Date: Dec. 6, 2018

(54) SPECTROSCOPY SYSTEM, LIGHT RECEIVING DEVICE, BIOLOGICAL INFORMATION MEASURING DEVICE, AND SPECTROSCOPY METHOD

(71) Applicant: SEIKO EPSON CORPORATION, Tokyo (JP)

(72) Inventor: Yasunori KOIDE, Matsumoto-shi (JP)

(73) Assignee: SEIKO EPSON CORPORATION, Tokyo (JP)

(21) Appl. No.: 15/972,640

(22) Filed: May 7, 2018

(30) Foreign Application Priority Data
May 31, 2017 (JP) 2017-108430

(51) Int. Cl. G01J 3/26; G01J 3/45; A61B 5/1455; A61B 5/145

(52) U.S. Cl. CPC G01J 3/26; G01J 3/45; A61B 5/14551; A61B 5/14532; A61B 5/1455

(57) ABSTRACT

A spectroscopy system includes: a spectral unit which selectively transmits light of a wavelength corresponding to one of a plurality of peaks of transmittance within a variable wavelength range; and a band pass unit which blocks light of a wavelength in a first range including apart of the plurality of peaks in the variable wavelength range and transmits light of a wavelength in a second range including another peak in the variable wavelength range.

### BACKGROUND

### 1. Technical Field

The present invention relates to a technique for spectrally dispersing light.

### 2. Related Art

JP-A-2012-127917 discloses a configuration to selectively detect light in a predetermined wavelength region. In the configuration disclosed in JP-A-2012-127917, a detection element detects light transmitted through a variable Fabry-Perot filter and a bandpass filter.

Specifically, in the configuration disclosed in JP-A-2012-127917, the variable Fabry-Perot filter transmits one of interfering beams of a plurality of orders and the bandpass filter transmits the interfering beam transmitted through the variable Fabry-Perot filter. The detection element detects the beam transmitted through the bandpass filter. The technique disclosed in JP-A-2012-127917 cannot generate the state where light is transmitted through neither the Fabry-Perot filter nor the bandpass filter because the transmission range of the bandpass filter coincides with the modulation band of the interfering beam transmitted through the variable Fabry-Perot filter.

### SUMMARY

An advantage of some aspects of the invention is that the state where a spectroscopy system transmits none of the wavelengths of light within a variable wavelength range (light shielding state) is generated.

A spectroscopy system according to an aspect of the invention includes: a spectral unit which selectively transmits light of a wavelength corresponding to one of a plurality of peaks of transmittance within a variable wavelength range; and a band pass unit which blocks light of a wavelength in a first range including a part of the plurality of peaks in the variable wavelength range and transmits light of a wavelength in a second range including another peak in the variable wavelength range. In this configuration, light of a wavelength in the first range including a part of the peaks in the variable wavelength range of the spectral unit is blocked, and light of a wavelength in the second range including another peak in the variable wavelength range is transmitted. Thus, the state where the spectroscopy system transmits none of the wavelengths of light within the variable wavelength range (light shielding state) can be generated.

In a preferred aspect of the invention, the first range is situated at an end on a short wavelength side or on a long wavelength side of the variable wavelength range. In this configuration, the first range is situated at the end on the short wavelength side or on the long wavelength side of the variable wavelength range. Thus, the configuration to transmit light in the second range is simplified, compared with a configuration where the first range is not situated at the end on the short wavelength side or on the long wavelength side of the variable wavelength range.

In a preferred aspect of the invention, the spectral unit transmits light of a wavelength corresponding to a peak corresponding to a voltage applied to the spectral unit, of the plurality of peaks, and the first range includes a peak occurring when no voltage is applied to the spectral unit. In this configuration, the first range includes a peak occurring when no voltage is applied. Thus, it is possible to reduce power consumption to generate the light shielding state. The invention can also be specified in the form of a method for spectrally dispersing light in the spectroscopy system with the foregoing configurations (spectroscopy method).

A light receiving device according to an aspect of the invention includes: the spectroscopy system according to one of the foregoing configurations; and a light receiving unit which generates a detection signal corresponding to a reception level of light transmitted through the spectroscopy system. In this configuration, a detection signal corresponding to the reception level of light transmitted through the spectroscopy system according to the foregoing configurations is generated. The spectroscopy system according to the foregoing configurations can generate the light shielding state. Thus, the light receiving device according to this configuration can generate a detection signal representing the state of the light receiving unit in the light shielding state, in addition to the detection signal corresponding to the reception level of the light transmitted through the spectroscopy system.

A biological information measuring device according to an aspect of the invention includes: a light emitting unit which emits light to a measurement site; the light receiving device according to the foregoing configuration which receives light transmitted through the measurement site; and a specifying unit which specifies biological information according to a detection signal generated by the light receiving device. The light receiving device according to the foregoing configuration can generate a detection signal representing the state of the light receiving unit in the light shielding state. Thus, the detection signal representing the state of the light receiving unit in the light shielding state can be used to specify biological information.

### BRIEF DESCRIPTION OF THE DRAWINGS

The invention will be described with reference to the accompanying drawings, wherein like numbers reference like elements.

FIG. 1 shows the configuration of a biological information measuring device according to a first embodiment of the invention.

FIG. 2 shows the configuration of a light receiving device.

FIG. 3 is an explanatory view showing the relation between transmittance characteristics of a spectral unit and transmittance characteristics of a band pass unit.

FIG. 4 shows the configuration of a light receiving device according to a second embodiment of the invention.

FIG. 5 is an explanatory view showing the relation between transmittance characteristics of a spectral unit and transmittance characteristics of a band pass unit according to a modification.

### DESCRIPTION OF EXEMPLARY EMBODIMENTS

### First Embodiment

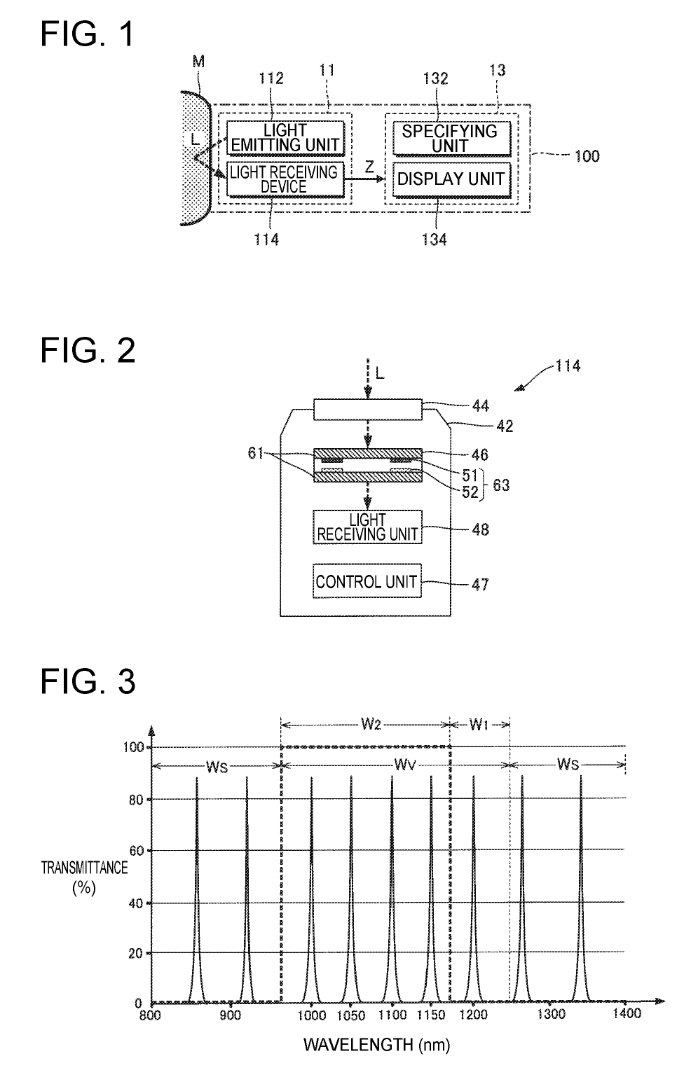

FIG. 1 shows the configuration of a biological information measuring device 100 according to a first embodiment of the invention. The biological information measuring device 100 of the first embodiment is a biological measuring instrument which non-invasively measures biological information of a user. For example, the concentration of various blood components of the user, such as blood sugar level (blood glucose concentration), hemoglobin concentration, or blood oxygen concentration, is a preferable example of biological information. In the first embodiment, blood sugar level is measured as biological information.

As illustrated in FIG. 1, the biological information measuring device 100 of the first embodiment includes an optical detection device 11 and an information processing device 13. The optical detection device 11 is an optical sensor module which generates a detection signal Z corresponding to the state of a site to be a measurement target (hereinafter referred to as a “measurement site”) M, of the user's body. The information processing device 13 specifies biological information of the user, based on the detection signal Z generated by the optical detection device 11.

As illustrated in FIG. 1, the optical detection device 11 has a light emitting unit 112 and a light receiving device 114. The light emitting unit 112 is a light emitting device which casts light L onto the measurement site M. Specifically, the light emitting unit 112 emits light L including near infrared light. The light emitting unit 112 in the first embodiment emits, for example, light L of 800 nm to 1400 nm. For example, the light emitting unit 112 is configured of a plurality of LEDs (light emitting diodes) which emit light in different wavelength regions from each other. However, the configuration of the light emitting unit 112 is not limited to this example.

The light L incident on the measurement site M from the light emitting unit 112 is diffused or reflected inside the measurement site M, then exits toward the light receiving device 114, and reaches the light receiving device 114 of FIG. 1. FIG. 2 shows the configuration of the light receiving device 114. The light receiving device 114 is an apparatus which receives the light L transmitted through the measurement site M. The light receiving device 114 has a casing 42, a band pass unit 44, a spectral unit 46, a control unit 47, and a light receiving unit 48. The casing 42 is a hollow structure formed of, for example, a light shielding material. An opening is formed on one face of the casing 42. The spectral unit 46, the control unit 47, and the light receiving unit 48 are accommodated inside the casing 42. The band pass unit 44 is installed in such a way as to close the opening of the casing 42. In the first embodiment, the light L transmitted through the measurement site M becomes incident on the band pass unit 44. The light L transmitted through the band pass unit 44, of the light L, is spectrally dispersed by the spectral unit 46. The spectral unit 46 is situated between the band pass unit 44 and the light receiving unit 48. That is, the spectral unit 46 is situated on the opposite side of the band pass unit 44 from the measurement site M.

The spectral unit 46 selectively transmits light within a specific wavelength region (hereinafter referred to as a “variable wavelength range”) WV. For example, a Fabry-Perot interferometer (etalon) is preferably used as the spectral unit 46. FIG. 3 shows the transmittance characteristics of the spectral unit 46 (relation between wavelength and transmittance). Specifically, the spectral unit 46 selectively transmits light of a wavelength corresponding to one peak (hereinafter referred to as a “transmission peak”) of a plurality of peaks of transmittance within a variable wavelength range WV. Here, the transmittance characteristics of the spectral unit 46 include peaks of transmittance corresponding to a plurality of different orders of interference. The variable wavelength range WV is, for example, a range where a peak corresponding to a specific order of interference exists in the transmittance characteristics of the spectral unit 46. In the first embodiment, a range where a plurality of peaks of transmittance for primary interference exists is described as the variable wavelength range WV. For example, a wavelength region of 950 nm or above and 1250 nm or below is the variable wavelength range WV. In FIG. 3, it is assumed that the plurality of peaks of transmittance is at the wavelengths of 1000 nm, 1050 nm, 1100 nm, 1150 nm, and 1200 nm in the variable wavelength range WV. In a range WS outside the variable wavelength range WV in FIG. 3, peaks of transmittance corresponding to other orders of interference than primary (for example, secondary interference) exist.

As illustrated in FIG. 2, the spectral unit 46 in the first embodiment includes a pair of reflection plates 61 facing each other, and an electrostatic actuator 63. Each reflection plate 61 is a plate-like half-transmission reflection member which transmits a part of incident light and reflects the other part. The electrostatic actuator 63 includes a first electrode 51 and a second electrode 52. The first electrode 51 is installed on one reflection plate 61. The second electrode 52 is installed on the other reflection plate 61. The distance between the reflection plates 61 changes according to the voltage value of a voltage (hereinafter referred to as a “control voltage”) applied between the first electrode 51 and the second electrode 52 from the control unit 47. Of the plurality of peaks of transmittance in the variable wavelength range WV, the transmission peak changes according to the distance between the reflection plates 61. That is, one of the plurality of peaks in the variable wavelength range WV is selected as the transmission peak according to the voltage value of the control voltage.

The control unit 47 controls the control voltage applied to the spectral unit 46. Specifically, the control unit 47 supplies the spectral unit 46 with a control voltage which changes within a range (hereinafter referred to as a “voltage range”) corresponding to the variable wavelength range WV. The voltage range corresponding to the variable wavelength range WV (950 nm to 1250 nm) is, for example, 0 V to 40 V. If the control voltage is high, the distance between the reflection plates 61 is short and the wavelength of the transmission peak in the variable wavelength range WV is short. Meanwhile, if the control voltage is low, the distance between the reflection plates 61 is long and the wavelength of the transmission peak in the variable wavelength range WV is long. For example, when the control voltage is 40 V, the wavelength of the transmission peak is 1000 nm. When the control voltage is 0 V (that is, when no voltage is applied between the electrodes), the wavelength of the transmission peak is 1200 nm. In the first embodiment, the control voltage is changed in time division to each of the voltage values of 40 V, 30 V, 20 V, 10 V, and 0 V. Thus, each of a plurality of peaks in the variable wavelength range WV is selected in time division as the transmission peak. As understood from the foregoing description, the spectral unit 46 transmits the light of the wavelength corresponding to the transmission peak corresponding to the control voltage applied to the spectral unit 46, of the plurality of peaks of transmittance in the variable wavelength range WV.

The band pass unit 44 of FIG. 2 is an optical filter which selectively transmits a component within a predetermined passband (wavelength region) and blocks other components. For example, a bandpass filter having a structure in which a plurality of transmission films with different refractive indexes is stacked is preferable as the band pass unit 44. As illustrated in FIG. 3, the variable wavelength range WV includes a first range W1 and a second range W2. The dashed lines in FIG. 3 show the transmittance characteristics of the band pass unit 44. As understood from FIG. 3, the band pass unit 44 transmits light of a wavelength in the second range W2, of the variable wavelength range WV. The band pass unit 44 blocks light of a wavelength in the first range W1, which is not in the second range W2, of the variable wavelength range WV, and light of a wavelength in the range WS outside the variable wavelength range WV. The first range W1 includes a part of the peaks in the variable wavelength range WV. The second range W2 includes the other peaks in the variable wavelength range WV. Specifically, the first range W1 is situated at the end on the long wavelength side of the variable wavelength range WV and includes a peak (wavelength of 1200 nm) generated when no control voltage is applied. Meanwhile, the second range W2 is a range other than the first range W1 of the variable wavelength range WV (specifically, a range on the short wavelength side as viewed from the first range W1) and includes all the other peaks (wavelengths of 1000 nm, 1050 nm, 1100 nm, and 1150 nm) than 1200 nm in the variable wavelength range WV. Specifically, the second range W2 transmitted by the band pass unit 44 is a range from 950 nm to 1175 nm. The second range W2 is broader than the first range W1.

As illustrated in FIG. 2, in the first embodiment, the light L transmitted through the measurement site M becomes incident on the band pass unit 44. The band pass unit 44 transmits the light in the second range W2 of the light L. The light in the second range W2 transmitted through the band pass unit 44 becomes incident on the spectral unit 46. The spectral unit 46 selectively transmits the incident light. The spectral unit 46 is controlled so as to be able to transmit, in time division, light of a wavelength corresponding to each of a plurality of peaks (wavelength of 1000 nm, 1050 nm, 1100 nm, 1150 nm, or 1200 nm) in the variable wavelength range WV. That is, the control unit 47 applies a control voltage in such a way that the spectral unit 46 can transmit the light of the wavelength corresponding to the peak in the first range W1, which is a light shielding target of the band pass unit 44, in addition to the light of the wavelength corresponding to each peak in the second range W2, which is a transmission target of the band pass unit 44. The light transmitted through the spectral unit 46 reaches the light receiving unit 48. As understood from the foregoing description, the band pass unit 44 and the spectral unit 46 function as a spectroscopy system which spectrally disperses the light L transmitted through the measurement site M.

The light receiving unit 48 generates a detection signal Z corresponding to the reception level of the light transmitted through the spectroscopy system. The detection signal Z is a signal representing, in time division, the intensity of the light of the wavelength at each peak in the variable wavelength range WV. For example, a light receiving element having a photoelectric conversion layer formed of InGaAs (indium gallium arsenide) showing a light receiving sensitivity to near infrared light is preferably used as the light receiving unit 48. The optical detection device 11 in the first embodiment is a reflection-type optical sensor module in which the light emitting unit 112 and the light receiving device 114 are situated on one side as viewed from the measurement site M.

The information processing device 13 of FIG. 1 is an apparatus to specify biological information from the detection signal Z generated by the light receiving device 114 of the optical detection device 11 and provide the biological information to the user. The information processing device 13 in the first embodiment has a specifying unit 132 and a display unit 134. The specifying unit 132 specifies biological information (blood sugar level) based on the detection signal Z generated by the light receiving device 114.

Here, there is a problem of a noise being superimposed on the detection signal Z, due to dark current generated in the light receiving unit 48 or external light such as sunlight or illumination light entering the casing 42. In the first embodiment, the light of the wavelength in the first range W1 of the variable wavelength range WV is blocked by the band pass unit 44. Therefore, when the transmission peak of the spectral unit 46 is within the first range W1 (that is, when the wavelength of the transmission peak is 1200 nm), it is the light shielding state, where none of the wavelengths of light in the variable wavelength range WV is transmitted through the spectroscopy system. That is, the reception level equivalent to the first range W1 of the detection signal Z indicates a noise due to dark current or external light. Thus, the specifying unit 132 specifies the intensity corresponding to the wavelength at each peak in the variable wavelength range WV from the detection signal Z and corrects the intensity corresponding to the wavelength at each peak in the second range W2, using the intensity corresponding to the wavelength at the peak in the first range W1. For example, the specifying unit 132 subtracts the intensity corresponding to the wavelength at the peak in the first range W1 from the intensity corresponding to the wavelength at each peak in the second range W2. The specifying unit 132 generates an absorption spectrum from the corrected intensity corresponding to the wavelength at each peak in the second range W2 and specifies the blood sugar level based on the absorption spectrum. To specify the blood sugar level using the absorption spectrum, for example, a known technique such as multiple regression analysis can be arbitrarily used. The multiple regression analysis may be, for example, PLS (partial least squares) regression analysis and independent component analysis or the like. The display unit 134 (for example, a liquid crystal display panel) displays the blood sugar level specified by the specifying unit 132.

As understood from the above description, the band pass unit 44 in the first embodiment blocks light of a wavelength in the first range W1 including a part of a plurality of peaks in the variable wavelength range WV of the spectral unit 46 and transmits light of a wavelength in the second range W2 including other peaks in the variable wavelength range WV. Therefore, the state where none of the wavelengths of light in the variable wavelength range WV is transmitted through the spectroscopy system (light shielding state) can be generated. With this configuration, the detection signal Z representing the state of the light receiving unit 48 in the light shielding state can be used to specify biological information. This enables highly accurate specification of biological information.

### Second Embodiment

In the first embodiment, the light L transmitted through the measurement site M becomes incident on the band pass unit 44, and the light L transmitted through the band pass unit 44, of the light L, is spectrally dispersed by the spectral unit 46. Meanwhile, in a second embodiment, the light L transmitted through the measurement site M becomes incident on the spectral unit 46, and a part of the light transmitted through the spectral unit 46, of the light L, is transmitted through the band pass unit 44.

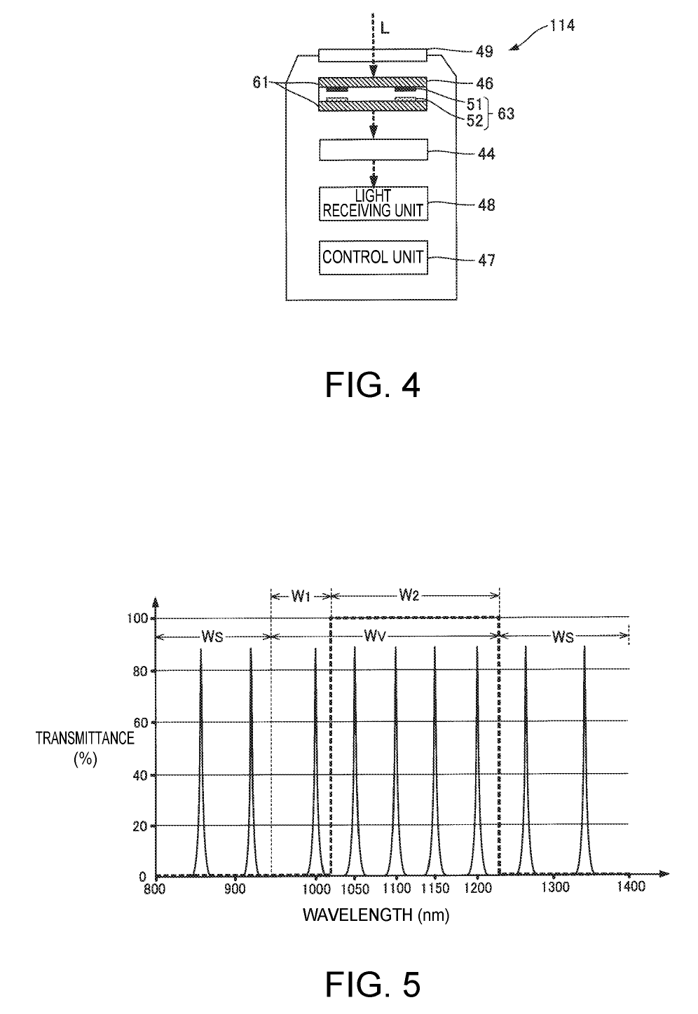

FIG. 4 shows the configuration of a light receiving device 114 according to the second embodiment. The light receiving device 114 has a casing 42, a band pass unit 44, a spectral unit 46, a control unit 47, and a light receiving unit 48, as in the first embodiment. The casing 42 in the second embodiment is a hollow structure, as in the first embodiment. A lid part 49 formed of a light-transmitting material is installed on one face of the casing 42. The other faces of the casing 42 are formed of a light-shielding material. As illustrated in FIG. 4, the band pass unit 44, the spectral unit 46, the control unit 47, and the light receiving unit 48 are accommodated inside the casing 42. The light transmitted through the measurement site M becomes incident on the spectral unit 46 via the lid part 49. In the second embodiment, the positional relation between the spectral unit 46 and the band pass unit 44 in the first embodiment is reversed. Specifically, the band pass unit 44 is situated between the spectral unit 46 and the light receiving unit 48. That is, the band pass unit 44 is situated on the opposite side of the spectral unit 46 from the measurement site M.

The optical characteristics of the spectral unit 46 and the band pass unit 44 are similar to those in the first embodiment. Specifically, the spectral unit 46 transmits, in time division, light of a wavelength corresponding to each (that is, a transmission peak) of a plurality of peaks (wavelength of 1000 nm, 1050 nm, 1100 nm, 1150 nm or 1200 nm) in the variable wavelength range WV, of the light L transmitted through the measurement site M. The light transmitted through the spectral unit 46 becomes incident on the band pass unit 44. The band pass unit 44 transmits the light in the second range W2, of the light transmitted through the spectral unit 46. The band pass unit 44 blocks light of a wavelength in the first range W1, which is not in the second range W2, of the variable wavelength range WV, and light of a wavelength in the range WS outside the variable wavelength range WV. The light of the wavelength in the second range W2 transmitted through the band pass unit 44 reaches the light receiving unit 48. As in the first embodiment, the light receiving unit 48 generates a detection signal Z corresponding to the reception level of the light transmitted through the spectroscopy system.

The information processing device 13 specifies biological information, based on the detection signal Z generated by the optical detection device 11, and provides the biological information to the user, as in the first embodiment. The specifying unit 132 of the information processing device 13 specifies the intensity corresponding to the wavelength at each peak in the variable wavelength range WV from the detection signal Z and corrects the intensity corresponding to the wavelength at each peak in the second range W2, using the intensity corresponding to the wavelength at the peak in the first range W1, as in the first embodiment.

As understood from the above description, in the second embodiment, the light of the wavelength in the first range W1 of the variable wavelength range WV transmitted through the spectral unit 46 is blocked by the band pass unit 44. Therefore, an effect similar to that of the first embodiment is realized. That is, when the transmission peak of the spectral unit 46 is within the first range W1 (that is, when the wavelength of the transmission peak is 1200 nm), it is the light shielding state, where none of the wavelengths of light in the variable wavelength range WV is transmitted through the spectroscopy system.

### Modifications

The embodiments described above can be modified in various ways. Specific examples of modification will be described below. Two or more modifications arbitrarily selected from the examples below can be properly combined.

(1) In the embodiments, a configuration in which the first range W1 is situated at the end on the long wavelength side of the variable wavelength range WV is described. However, the position of the first range W1 is not limited to this example. For example, a configuration in which the first range W1 is situated at the end on the short wavelength side of the variable wavelength range WV as illustrated in FIG. 5 can be preferably employed. Also, a configuration in which the first range W1 is situated in the middle of the variable wavelength range WV may be employed. However, the configuration in which the first range W1 is situated at the end on the short wavelength side or the long wavelength side of the variable wavelength range WV simplifies the configuration to transmit the light in the second range W2, compared with the configuration in which the first range W1 is situated in the middle of the variable wavelength range WV. Also, in the configuration in which the first range W1 is situated at the end on the short wavelength side or the long wavelength side of the variable wavelength range WV, the first range W1 is connected to the range WS on the short wavelength side or the long wavelength side as viewed from the variable wavelength range WV. Therefore, there is no need to separately provide an element to block light of a wavelength in the first range W1 and an element to block light of a wavelength in the range WS. This simplifies the configuration of the spectroscopy system.

(2) In the embodiments, a configuration in which the band pass unit 44 blocks light of a wavelength in the first range W1, which is not in the second range W2, of the variable wavelength range WV, and light of a wavelength in the range WS outside the variable wavelength range WV, is described. However, the range of wavelength of light to be blocked by the band pass unit 44 is not limited to this example. For example, if the light emitting unit 112 emits light L of a wavelength in the variable wavelength range WV (for example, if the light emitting unit 112 emits light L of 950 nm to 1250 nm), the configuration in which the band pass unit 44 blocks light of a wavelength in the range WS is not essential. As understood from the above description, whether the band pass unit 44 blocks light of a wavelength outside the first range W1 or not may be arbitrarily decided, provided that the band pass unit 44 can block light of a wavelength in the first range W1 including a part of peaks in the variable wavelength range WV.

(3) In the embodiments, the range where light of a specific order of interference exists is defined as the variable wavelength range WV. However, a part of the range where light of a specific order of interference exists may be defined as the variable wavelength range WV.

(4) In the embodiments, the first range W1 is situated at an end (end on the long wavelength side) of the variable wavelength range WV and includes a peak occurring when no control voltage is applied. However, the relation between the wavelength of each peak in the variable wavelength range WV and the control voltage is not limited to this example. For example, it is not essential that the first range W1 including a peak occurring when no control voltage is applied is situated at an end of the variable wavelength range WV. Also, a configuration in which the first range W1 includes a peak occurring when a control voltage is applied can be employed. However, with the configuration in which the first range W1 includes the wavelength of a peak occurring when no control voltage is applied, the power consumption to generate the light shielding state can be reduced regardless of whether the first range W1 is situated at an end (end on the long wavelength side) of the variable wavelength range WV or not.

(5) In the embodiments, a configuration in which the first range W1 includes one peak of a plurality of peaks in the variable wavelength range WV and in which the second range W2 includes all the other peaks is described. However, the number of peaks included in the first range W1 and the second range W2 is not limited to this example. For example, a configuration in which the first range W1 includes two or more peaks, or a configuration in which the second range W2 includes a part of a plurality of peaks that is not included in the first range W1 can be employed.

(6) In the embodiments, light of a wavelength corresponding to each peak (that is, transmission peak) of a plurality of peaks (wavelengths of 1000 nm, 1050 nm, 1100 nm, 1150 nm, and 1200 nm) in the variable wavelength range WV is transmitted in time division. However, the light of the wavelength corresponding to each of the plurality of peaks in the variable wavelength range WV can be transmitted in time division in an arbitrary order. For example, the light of the wavelength corresponding to each of the plurality of peaks (wavelengths of 1000 nm, 1050 nm, 1100 nm, and 1150 nm) included in the second range W2 and the light of the wavelength corresponding to the peak (wavelength of 1200 nm) included in the first range W1 may be transmitted alternately. Specifically, light corresponding to the wavelengths at the peaks is transmitted in the order of 1000 nm, 1200 nm, 1050 nm, 1200 nm, 1100 nm, 1200 nm, 1050 nm, and 1200 nm, and a detection signal Z is thus generated. The specifying unit 132 detects an intensity corresponding to the wavelength at each peak in the variable wavelength range WV, based on the detection signal Z, and corrects the intensity corresponding to wavelength at each peak in the second range W2, using the intensity corresponding to the wavelength at the peak in the first range W1 immediately after the wavelength at each peak in the second range W2. This configuration enables more accurate correction of the intensity corresponding to the wavelength at each peak in the second range W2, than the configuration in which the light of the wavelength corresponding to the peak included in the first range W1 is transmitted after the light of all the wavelengths corresponding to the plurality of peaks included in the second range W2 is transmitted.

(7) In the embodiments, the biological information measuring device 100 displays biological information. However, the display of biological information is not essential in the biological information measuring device 100. For example, it is possible to transmit biological information specified by the specifying unit 132 to a terminal device (for example, a smartphone) capable of communicating with the biological information measuring device 100 and cause the display unit 134 of the terminal device to display the biological information. That is, the display unit 134 can be omitted from the biological information measuring device 100. Also, a configuration in which the terminal device is provided with one or both of the specifying unit 132 and the display unit 134 can be employed. For example, the specifying unit 132 is implemented by an application executed on the terminal device. As understood from the above description, the biological information measuring device 100 can also be implemented by a plurality of devices configured separately from each other.

(8) The invention can also be specified as a spectroscopy method for a spectroscopy system. Specifically, a spectroscopy method according to a preferred embodiment of the invention includes: selectively transmitting light of a wavelength corresponding to one of a plurality of peaks of transmittance in a variable wavelength range; and blocking light of a wavelength in a first range including a part of the plurality of peaks in the variable wavelength range, and transmitting light of a wavelength in a second range including another peak in the variable wavelength range.

The entire disclosure of Japanese Patent Application No. 2017-108430 is hereby incorporated herein by reference.

What is claimed is:

1. A spectroscopy system comprising:
a spectral unit which selectively transmits light of a wavelength corresponding to one of a plurality of peaks of transmittance within a variable wavelength range; and
a band pass unit which blocks light of a wavelength in a first range including a part of the plurality of peaks in the variable wavelength range and transmits light of a wavelength in a second range including another peak in the variable wavelength range.

2. The spectroscopy system according to claim 1, wherein
the first range is situated at an end on a short wavelength side or on a long wavelength side of the variable wavelength range.

3. The spectroscopy system according to claim 1, wherein
the spectral unit transmits light of a wavelength corresponding to a peak corresponding to a voltage applied to the spectral unit, of the plurality of peaks, and
the first range includes the peak occurring when no voltage is applied to the spectral unit.

4. A light receiving device comprising:
the spectroscopy system according to claim 1; and
a light receiving unit which generates a detection signal corresponding to a reception level of light transmitted through the spectroscopy system.

5. A light receiving device comprising:
the spectroscopy system according to claim 2; and
a light receiving unit which generates a detection signal corresponding to a reception level of light transmitted through the spectroscopy system.

6. A light receiving device comprising:
the spectroscopy system according to claim 3; and
a light receiving unit which generates a detection signal corresponding to a reception level of light transmitted through the spectroscopy system.

7. A biological information measuring device comprising:
a light emitting unit which emits light to a measurement site;
the light receiving device according to claim 4 which receives light transmitted through the measurement site; and
a specifying unit which specifies biological information according to a detection signal generated by the light receiving device.

8. A spectroscopy method comprising:
selectively transmitting light of a wavelength corresponding to one of a plurality of peaks of transmittance in a variable wavelength range; and
blocking light of a wavelength in a first range including a part of the plurality of peaks in the variable wavelength range, and transmitting light of a wavelength in a second range including another peak in the variable wavelength range.
